(19) United States
(12) Patent Application Publication
(10) Pub. No.: US 2006/0149296 A1
(43) Pub. Date: Jul. 6, 2006

(54) OR ready

(76) Inventor: Sydney Delvin Stanners, Sidney (CA)

Correspondence Address: Sydney Delvin Stanners, 203 - 9901 4th Street, Sidney, BC, U8L 2Z6, CA

(21) Appl. No.: 11/253,607

(22) Filed: Oct. 20, 2005

Related U.S. Application Data
(60) Provisional application No. 60/619,942, filed on Oct. 20, 2004.

(51) Int. Cl. A61B 17/00

(52) U.S. Cl. 606/116

(57) ABSTRACT

An apparatus and method for preventing wrong-site surgery is designed to enable physicians, nurses and a patient to quickly and accurately select and record the patient diagnosis, procedure and correct anatomical site of their patient's upcoming surgery. The patient data is viewed by the surgical team on an operation monitor before beginning surgery to prevent wrong-site surgery.

This application claims the benefit of the filing date of Provisional Patent Application. No. 60/619,942 Filing Date Oct. 20, 2004 Title: OR READY

### SUMMARY

Patients who suffer wrong-site surgery are not the only ones devastated by this preventable medical error—the surgeon, the surgical team, and the patients' family is deeply affected, too. Add to this the high costs of mal practice litigation faced by the surgeon and hospital, and the costs—measured in both human trauma and financial payouts—quickly escalate.

‘Wrong site-surgery’ is a catchall phrase that describes the following surgical procedures performed on a patient [in error]:*
*Hi-lights from AORNs' Position Statement on Correct-Site Surgery—see page 14 for article.

Surgery on the wrong patient.
On the wrong body part.
On the wrong side of the body.
At the wrong level of a correct site.

The Joint Commission on Accreditation of Healthcare Organizations (JCAHO) considers all wrong-site surgeries—no matter to what degree—to be sentinel events**, and reviewable by a JCAHO committee.
**Sentinel event “A sentinel event is an unexpected occurrence involving death or serious physical or psychological injury, or the risk thereof. Serious injury specifically includes loss of limb or function. The phrase ‘or risk thereof’ includes any process variation for which a recurrence would carry a significant chance of serious adverse outcome. Such evens are called “Sentinel” because they signal the need for immediate investigation and response.”

Wrong site-surgeries occur for many reasons. Two of the most prevalent reasons are:
Poor communication between the surgeon, surgical team, and the patient.

Communication problems are not surprising given the conditions prevalent in today's hectic hospital scenario—with budget restraints and too few people doing too much in terms of providing adequate patient care. This results in many caregivers working excessive hours, which in turn ultimately leads to diminished work performance and mistakes.
Lack of specific institutional guidelines.

Above all others, lack of specific institutional guidelines [which would provide a detailed course of action covering the patients' pre op work-up] is almost certainly responsible for more wrong-site surgeries occurring than any other set of—error causing—circumstances.

A well-planned—and diligently carried out—pre op, correct-site guideline would most certainly circumvent virtually all wrong-site errors from progressing to the critical stage of causing harm to the patient.

Many excellent suggestions are being put forward in an attempt to prevent wrong-site errors. However, these tend to be fragmented approaches that would see one set of guidelines used in hospital A, another set of guidelines used in hospital B, and yet another used in hospital C etc. Surgical team members, moving from one jurisdiction to another, and following their home hospital rules, might find these very rules lead to errors in a new jurisdiction.

There is an urgent need for North American hospitals to adopt a standardized approach that will see all OR teams using the same specific guidelines. Such an approach would have various far-reaching benefits, and above all, would arm the OR team members [and other patient stakeholders] with a clearly defined set of actions. In other words, each team member/stakeholder would know exactly what is expected of him or her, leaving no chance for mix-ups or over-sights.

The following article, reprinted from the New York Times, provides an insight into wrong-site surgeries.
Dec. 11, 2001
The Wrong Foot, and Other Tales of Surgical Error
By: Lawrence K. Altman, M. D.
Most people can easily tell right from left, but for some surgeons it seems to be a problem.
At least 150 times since 1996, surgeons in hospitals in this country have operated on the wrong arm, leg, eye, kidney, or other body part, or even on the wrong patient. The figure does not include near misses—when surgeons started to operate on the wrong site or patient—because no one collects such information.
Operations on the wrong site or the wrong patient should never happen, but too often they do, an alarmed Joint Commission on Accreditation of Healthcare Organizations said last week, in issuing its second alert in three years aimed at reducing the number of surgical mistakes.
Because the first alert failed to stop the errors, Dr. Dennis S. O'Leary, the commission's president, said his organization tried “a different tact, this time directed at patients,” urging them to demand that their surgeons mark the surgical site before going to the operating room. Or, Dr. O'Leary said, patients should do it themselves.
He urged patients to mark not only the site that is to be operated on, but also the one that should not be touched. “We have had two cases where the correct limb was marked but the wrong one was not, and, of course, the surgeon never went to the right one so operated on the wrong one,” Dr. O'Leary said.
The commission's action reflects and unsettling fact that doctors and patients may prefer not to think about—that serious mistakes can happen even in the best of hospitals, and stronger efforts are needed to prevent them.
The 150 mistakes mostly concerned arms, legs, and paired organs and occurred in only a tiny fraction of the millions of operations performed since 1996. Nevertheless, “the tolerance here is zero,” Dr O'Leary said.
Reasons for the errors are many and include similar sounding names, failure to check patient names on medical records and reversing the sides of X-rays and scans placed on viewing boxes in the operating room.
In addition to saying the surgical site should be marked, the commission made these two other recommendations: In the operating room before starting surgery, all members of the surgical team should confirm that they have the correct patient, surgical site and procedure. And, the operating room team should take “a timeout” to check medical records and X-rays, discuss among themselves what they are about to do, and corroborate information with the patient.
The alert came on the same day that the New York State Health Department disclosed that it had fined Long Island College Hospital in Brooklyn $124,000 because two of its neurosurgeons operated on the wrong side of a patient's brain last February.
It was the second reported error at the hospital since June 2000, when an ophthalmologist began operating on the wrong eye of a patient.
Other types of surgical mistakes also occur. Last month in Seattle, the University of Washington accepted responsibility when one of its surgical teams left 13-inch instrument in a patient after removing a large cancer from his abdomen.
Widespread publicity was given to a wrong-site brain operation at Memorial Sloan-Kettering Cancer Institute on Manhattan in 1995.
In 1999, the Institute of Medicine of the National Academy of Sciences and other national organizations drew attention the problem, saying they were concerned that patients' safety may have been compromised by cost containment measures and the increase in the number of out-patient operations performed at ambulatory surgical centers.
The errors have also come to light because as the public has paid the medical bills under the federal Medicare and other health insurance plans, the government and researchers can now study information that could not be collected in decades past. And consumer awareness of health care issues has increased.
New York says it is one of the few states to have taken aggressive action to promote patient safety. In February, the state health department issued recommendations similar to the Joint Commission's aimed at preventing operations on the wrong body sites and the wrong patients.
The Joint Commission said most wrong-site surgery errors were reported by hospitals where the mishaps took place, with the remaining cases coming from patient complaints and news articles. Reports are mainly voluntary, though penalties may be imposed for not reporting them in certain instances. Much of the information is kept confidential.
Releasing that information would be “a litigator's dream,” said Charlene D. Hill, a spokeswoman for the Joint Commission. “You would be basically writing the legal brief.”
An additional concern, she said, is that disclosing the names of doctors and hospitals may lead them to hide subsequent errors.
The New York State Health Department publicly discloses the names of hospitals, but not patients, after it investigates and fines institutions.
In a report released last week, the department cited a number of violations related to the wrong-site brain surgery at Long Island College Hospital.
One was a lack of communication among the surgeons, anesthesiologist and circulating nurse. Another was that the hospital allowed an unlicensed doctor on the neurosurgical team to be first assistant in the operation.
The victim of the mistake was a patient with bleeding in one side of his skull, in the area between the skull bone and the lining covering the brain, from a condition known as an epidural hematoma. Despite the emergency, the state said, an hour and a half passed before neurosurgeons began operating on the patient.
When the doctors did finally get to the bleeding patient, they initially began operating on the wrong side of his skull. When they recognized the error, they switched to the correct side. Although the patient did not suffer any adverse effects from the surgery, beyond a hole on the wrong side of his skull, “there was potential for severe neurological deficit or even death,” the state said.
The state said that the hospital took acceptable corrective action regarding the two neurosurgeons. One resigned, and the other's privileges were not renewed, a hospital spokesman said.
At the University of Washington Medical Center, officials said surgeons had never performed wrong-site surgery, but they had left instrument in patients.
In June, Dr. David Byrd's team did not notice that it had left a long instrument in a patient, Donald Church, after removing a large cancer from his abdomen. The instrument is designed as a shield to help prevent a surgeon from accidentally sewing a piece of bowel to abdominal muscles in closing the incision.

Surgical Associations and Medical Jurisdictions have recently issued statements on their position on wrong site-surgery. Since it is beyond the scope of this paper to include all statements, the following two serve to encapsulate the concern shown by all stakeholders.

New York State Health Department Releases Pre-Operative Protocols to Enhance Safe Surgical Care

New York State Health Department Releases Pre-Operative Protocols to Enhance Safe Surgical Care

State of New York Department of Health

ALBANY, Feb. 8, 2001—The New York State Department of Health released recommendations from its Pre-Operative Protocols Panel as part of a statewide effort to further safeguard patients' care during surgical procedures.
“New York State is committed to providing residents access to quality health care in a cost effective manner and paramount in that is patient safety,” said State Health Commissioner Antonia C. Novello, M. D., M. P. H., Dr. P. H. “in working with the Governor to reduce medical and surgical errors, we believe very strongly that enhancing communication among health care providers and increasing practitioner awareness combined with strong provider protocols will aid in increasing patient safety measures currently in place.”
Dr. Novello appointed the 12-member panel, including nine physicians and three registered nurses, last June in response to increased scrutiny of proper patient care in our nation's hospitals and the Department's detection of some critical patient care errors including: the wrong patient in the operating room, surgery performed on the wrong site or side, wrong procedure performed, failure to communicate patient condition changes, disagreements regarding stopping procedures and failure to communicate/report errors.
After reviewing policies and procedures from a variety of hospitals and protocol and guideline information from physician and nursing associations, other states, the Internet and the Institute of Medicine report of 2000 and listening to public input, Commissioner Novello adopted these recommendations as a standard of care for hospitals to ensure safe patient care outcomes and avoid these surgical errors in a variety of patient care settings-.
   Hospitals should develop and implement policies and procedures to assure there are at least three independent verifications of the surgical site, location and correct patient identification.
   The attending physician should sign the consent form prior to the induction of anesthesia, confirming the accuracy of the document including the description of the procedure.
   As one of the three independent verifications, it is recommended that the surgeon of record mark or unequivocally identify the site and/or side prior to surgery. The marking technique should be determined by the facility.
   Whenever possible, the surgeon of record or his/her designee, should physically see and talk to the patient in the peri-operative area on the day of surgery.
   When laterality (the procedure is specific to one side of the body) is at issue, the words or should be spelled out in their entirety, on the operative schedule and the operative consent form.
   The anticipated level(s) for spinal surgery should be indicated on the operative schedule and the operative consent form. Levels may be modified later if operative findings indicate differences.
For operating room settings (for other settings, use appropriate personnel), the circulating nurse will:
   Ensure the correct patient is present;
   The consent has been signed by the surgeon of record on the day of surgery;
   The appropriate surgical side/site has been identified/marked;
   The surgeon has selected for display appropriate and relevant radiological films for the planned procedure (the surgeon of record determines what is appropriate and relevant); and
   Ensure there is agreement as to the planned procedure, which has been verified with the surgeon, anesthesia personnel and circulating nurses. The agreement must be documented in the medical record.
Another key area that was discussed was how best to maintain open lines of communication among surgical team members and with patients. It was agreed that facilities should define the responsibilities of each staff member involved with the procedure and that there should be verbal communication regarding consent, marking, and/or appropriate equipment and supplies. The panel stresses the need for the surgeon, anesthesia personnel, and the circulating nurse to discuss patient issues and the planned procedure prior to the commencement of the procedure to ensure that all are familiar with the strategies and expectations for conducting the procedure; any special issues or potential problems should be identified and discussed. To further enhance communication, the patient's medical record should be fully documented.
The panel calls for each health care facility to have a policy on dispute resolution that should address such areas as discussions between physicians, failure to disseminate critical information, conversations whether or not to continue a procedure and communications between nurses and physicians. According to the panel, if there are any discrepancies in information or disagreements regarding the procedure/equipment/supplies, the surgical procedure is to be delayed until the issues are resolved.
Hospitals and other health care facilities throughout the State are expected to have a policy, which at the very least, includes these issues. The facilities can add to the guidelines and implement them in the manner best suited to their operation. Once implemented, they will assist medical personnel in providing high quality and safe patient care and in bringing uniformity to the patient care process.

Please refer to Appendix for the following article.

The Association of Perioperative Nurses (AORN) has taken a strong stance on preventing wrong-site surgery. Perioperative nurses work with surgeons who represent all surgical specialties, and are therefore privy to the causes of wrong-site surgery in all operating room (OR) settings and procedures.

Error Prevention—a Learning Curve

Faced with a similar set of problems as those now facing the medical profession in preventing wrong-site surgeries, aviators developed, and now use, a very successful checklist system which reduces aviation accidents, especially those caused by pilot error. The checklist is in use by pilots internationally.

Several striking analogies exist between medical and aviation professionals:
Both are well trained and highly competent.
Both groups exhibit the human frailty of occasionally making mistakes.
The media treats these mistakes—especially the ones resulting in serious injury or death—with a great amount of sensationalism.

When it comes to preventing mistakes, aviation professionals have a lengthy track record of “having been there—done that”, and can be considered expert in their approach to understanding and preventing accidents. Through the years, aviators have developed a very successful standard checklist—with proven results.

The aviation industry has long understood the power of the Checklist in preventing accidents—especially those caused by “pilot error”. Pilots who fly small two seat Cessna's, light twins, biz jets or 747's etc. in Europe, Asia, Australia or in any other part of the world, all share one thing in common—they rigidly adhere to using a standardized written checklist (specifically written for the airplane they are flying). Pilots must refer to their checklists before taking off and before landing. There are no exceptions to this rule. Using a written checklist is one of the first lessons a student pilot must learn, and become proficient with before h/she is allowed to solo. The aviation industry has learned—through a sustained learning curve—that small errors, undetected before a flight commences, during flight, or before a landing is initiated, can have drastic outcomes. Imagine, if you will, that all pilots worldwide, decided that tomorrow, they would not use their checklists . . . . ? Undoubtedly, accident investigators would be required to work overtime for many months to come.

Putting an End to Wrong-Site Surgery

There is no need to reinvent the wheel to assure that the patient is the recipient of correct-site surgery. By following the lead—and very successful checklist system—developed and used by the aviation industry, OR teams can expect an ongoing 100% correct-site surgery performance rate.

Putting an end to wrong-site surgery can easily be achieved by using a customized patient checklist that definitively establishes four (4) specific patient OR criteria:
The right patient.
The right site
The right procedure.
The right outcome

Since it is untenable to rely on a single team member (i.e. the surgeon, for example) to be the sole source of patient data, the checklist requires that no less than three patient/OR stakeholders [independently] input the necessary patient pre-surgery information. Average time to input data is one minute.

Checklist Stakeholders

Checklist stakeholders are those individuals who have intimate knowledge [or are privy to that knowledge] of the patients' upcoming surgery, and as such, can influence the outcome of certain aspects of the surgery. Therefore, those qualified to select and input correct-site surgery checklist criteria would include—but are not limited to—the following:
The GP
The Specialist
The Surgeon
The Circulating Nurse
The Patient*
The Patients' Family or Significant Other*
   *These Stakeholders use a different method to indicate the correct site.

The success of the checklist is based on the premise that while one individual might err in h/her selection of the correct surgical site (or procedure), the chances of three or four knowlegible stakeholders [independently] selecting the wrong site—while examining the patient, or referring to the patients' charts—would be astronomically high. Furthermore, given that all members of the surgical team will view the patients' [customized] checklist immediately before the surgical procedure begins, reduces the risk of wrong-site surgery virtually to zero.

### BRIEF DESCRIPTION OF THE DRAWINGS

FIG. 1 is an example of displaying basic anatomical detail for a patient in accordance with an embodiment of the invention.

FIG. 2 is a block diagram of a menu in accordance with an embodiment of the invention.

FIG. 3a-3c is a chart of a patient in accordance with an embodiment of the invention.

FIG. 4 is a block diagram of a menu in accordance with an embodiment of the invention.

### OR READY™

OPERATING ROOM READY™ (OR READY™) is the trade name of the Company's checklist

The Company's OR READY™ approach to correct-site surgery provides the surgical team a succinctly detailed outline to the patients' surgery at the time its' needed most—in the OR prior to beginning the procedure.

OR READY™

The following inventions relate to a unique software program that enables the physician and nurse stakeholders to quickly and accurately select and record the patient diagnosis, procedure and correct anatomical site of their patients' upcoming surgery. This data, together with the patients' identity work-up, is viewed by the surgical team on an OR monitor immediately before beginning surgery.

How it Works

The patient purchases an OR READY™ kit from h/her GP, local pharmacy or through the web at www.orready.com for a nominal fee—i.e. $9.95. The kit consists of:
A floppy disc.
Instruction pamphlet.
Single (1) tattoo stencil.
   A Fee Based Service

The patient takes the OR READY™ disc on each appointment related to h/her upcoming surgery—i.e. G.P., specialist and surgeon. Using their PC—or PDA with OR READY software—each physician selects the patients' diagnosis, procedure and surgical site (if using a PDA, the patient data is downloaded to the PC and transferred to the OR READY checklist disc). When the patient attends the G.P. for their pre op work-up, the G.P. completes h/her Stakeholder part of the program, and the doctors' nurse completes both the I.D. and medical history portion of the program—a fee of $20 etc. is charged to the patient for the nurses time. The OR READY disc is left with the G.P., and is included with the pre op report that is sent to the hospital, and subsequently accompanies (the patient file) to the OR. On the day of the operation, the circulating nurse discusses the surgery with the patient, and after the interview, completes the circulating nurses' Stakeholder portion of the OR READY™ program. Just as the patient enters the OR, the circulating nurse readies the surgical team to take a ‘time out’ to view the patients' OR READY™ checklist on an OR monitor, and checks that the patients' TATTOO matches the site(s) depicted on the OR READY chart.

Patient Responsibility

It is the responsibility of the patient (or the patients' significant other etc.) to affix the TATTOO over the site of the surgery. This procedure should be carried-out the day before surgery, and is fully explained in the INSTRUCTION PAMPHLET.

The Software Program

The OR READY™ floppy disk is inserted into a PC (if using a PDA, the data is subsequently downloaded to the PC/disc)

The physician(s), using their PC or PDA, enters the anatomical body part or organ that is selected to undergo surgery. For example, a diabetic patient, about to undergo amputation of the left foot, would benefit from the following safety protocol that protects the right foot—and other body parts—from wrong-site [wrong-side] surgery.

The physician follows the OR READY™ program, which—after entering FOOT*—provides a series of menus' that are related to foot surgeries. The physician simply ‘points and clicks’ on the correct response:
*The program gives the user the option of choosing the ticular ANATOMY system relative to their patient diagnosis—in which case—site selection is obtained by the point and click method.

Enters FOOT
   Prompted to select RIGHT or LEFT foot
Clicks on LEFT foot
   Prompted to select DIAGNOSIS
Clicks on GANGRENE
   Prompted to select PROCEDURE
Clicks on AMPUTATE

OR READY™ automatically arranges the patient data in three (3) charts that are subsequently viewed on an OR monitor screen by the surgical team, in the ‘time out’ period, before beginning the patients' surgery.

Note: an additional chart(s) may be necessary in some cases.

Program Detail

OR READY's program enables the physician Stakeholder to quickly select and display any part of the human anatomy and describe the appropriate surgery to match the patient diagnosis. Entering the necessary OR READY patient data, takes only moments, and the payback is substantial—protection of the patient from injury or death due to wrong-site surgery errors.

The OR READY program is accessible at two levels:
Level I displays basic anatomical detail and concise patient history.
Level II gives a detailed anatomical chart printout—the physician chooses Level II or I.
Level II provides accesses to the next layer of patient history.

The physician has the option of selecting portions of Levels I and II. For example, the physician may wish to use Level I basic anatomy together with Level II history etc.

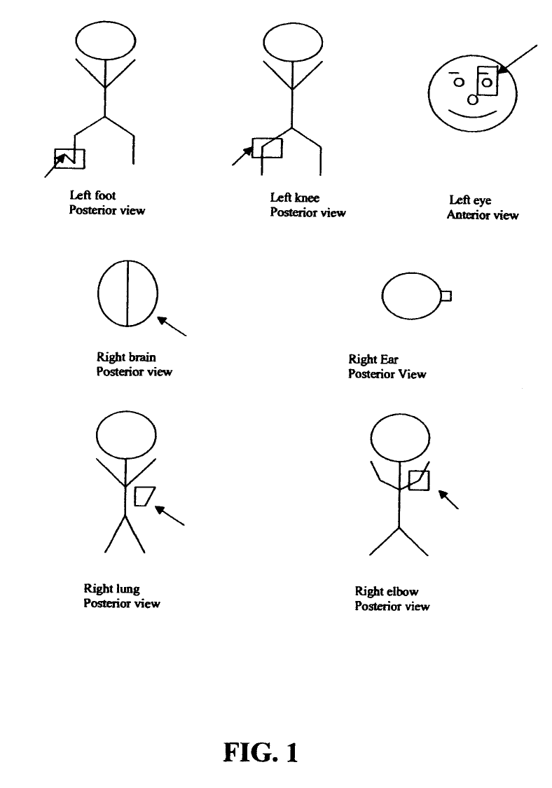

Level I anatomical detail examples are illustrated in FIG. 1

Independent Selection

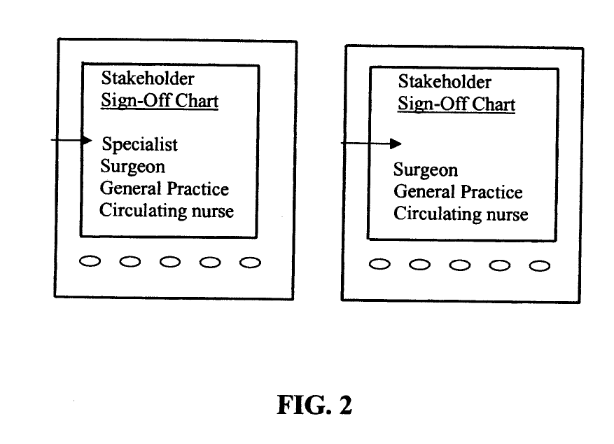

Each medical Stakeholder inputs OR READY™ patient data, based on h/her direct knowledge of the case. As each medical specialty entry is completed, that specialty is removed from the menu, (see FIGS. 1 and 2) and the entry cannot be viewed until the circulating nurse completes h/her patient input and is prompted to check “DONE”. The 3 OR READY™ charts are now ready for viewing by the surgical team in the ‘time out’ period, immediately before beginning the surgical procedure.

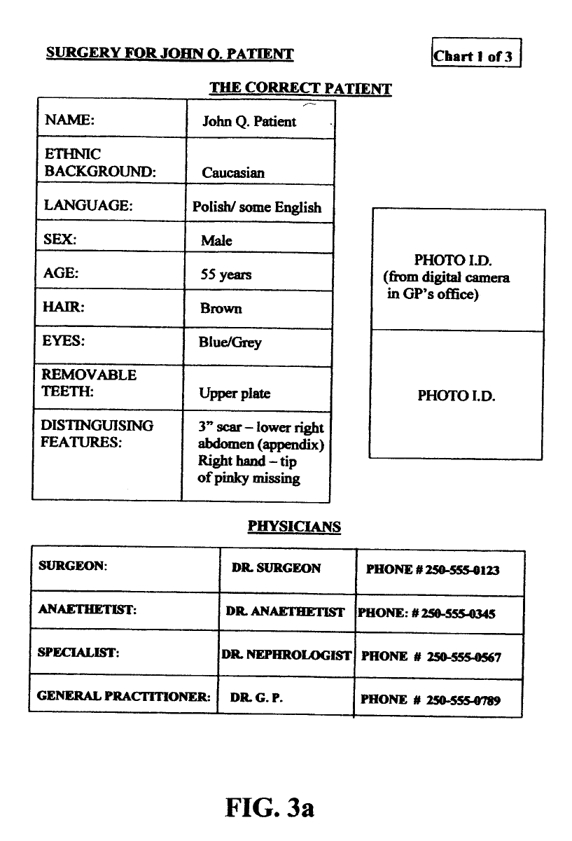

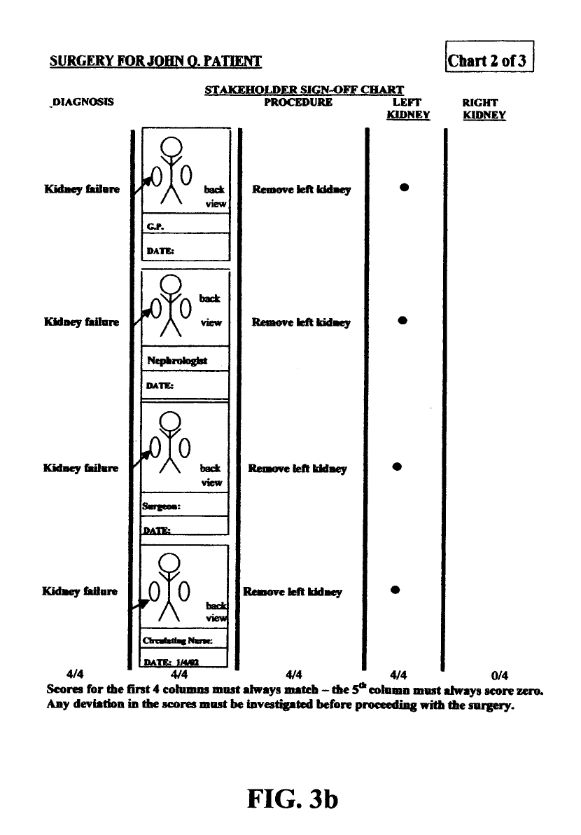

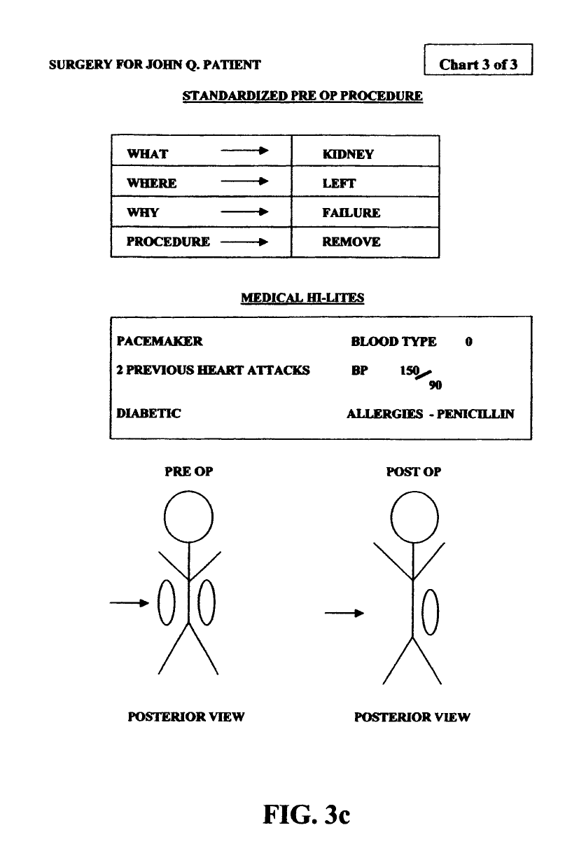

FIG. 3a, 3b, 3c illustrate charts which are viewed by the Surgical team or an OR monitor before proceeding with the surgical procedure.

Transplants

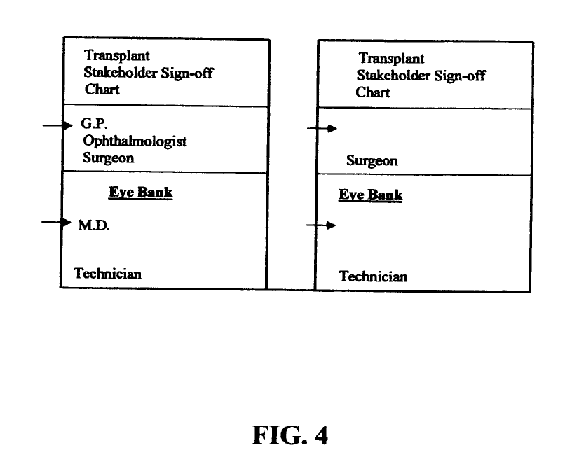

FIG. 4 illustrates that patients who are about to undergo transplant surgery are requried to purchase an additioal disc—TRANSPLANT READY™, TRANSPLANT READY™ is viewed in the OR in conjunction with OR READY™ in sequence.

### CONCLUSION

The OR READY checklist is an indispensable surgical team tool, specifically designed as user friendly to both customize and use, and presents the patients' data in a concise and visually pleasing format. Use of OR READY in North American ORs' will see an abrupt end to wrong-site surgeries.

OR READY is designed as a standard international protocol

AORN Position Statement on Correct Site Surgery

Preamble

The Institute of Medicine's (IOM) report To Err is Human: Building a Safer Health System has brought national attention to the necessity to improve patient safety. 1 A comprehensive approach is needed in each health care delivery system to prevent wrong site surgery. Procedures and protocols should be developed collaboratively by multidisciplinary teams, including surgeons, perioperative RNs, anesthesia care providers, risk managers, and other health care professionals. Perioperative RNs should be key participants in multidisciplinary teams as they develop these procedures and protocols. As patient advocates, perioperative RNs have a duty to the public to protect the patient from injury and to safeguard the patient's health, welfare, and safety. 2 A central goal of perioperative nursing is to assist patients in achieving a level of wellness equal to or greater than that which they had before surgical intervention. While it is the surgeon's responsibility to diagnose the patients need for surgery and to delineate the surgical site, verifying the correct surgical site at the time of surgery is the responsibility of each health care provider, including perioperative RNs—

Background

Wrong site surgery is a broad term that encompasses all surgical procedures performed on the wrong patient, wrong body part, wrong side of the body, or at the wrong level of the correctly identified anatomic site. 3 The Joint Commission on Accreditation of Healthcare Organizations (JCAHO) considers all wrong site surgeries, regardless of the extent of the procedure, to be sentinel events. As such, they are reviewable under the JCAHO sentinel event procedure. 4 This procedure calls for a root cause analysis of each sentinel event. Review of several root cause analyses by the JCAHO Accreditation Committee of the Board of Commissioners found wrong site surgery most commonly occurs during orthopedic procedures, followed by urologic and neurosurgical procedures. 5

Recognizing that wrong site surgery is most common in orthopedic procedures, the American Academy of Orthopedic Surgeons (AAOS) is committed to eliminating the incidence of wrong site surgery. The AAOS has developed a ‘Wrong-Site Surgery Advisory Statement’ in which it notes that it is the surgeon's responsibility to identify and mark the correct surgical site. 6 Recognizing that wrong site surgery, is not only an orthopedic problem, the AAOS has called for a comprehensive effort by other surgical specialties and health care professionals in developing protocols to effectively eliminate wrong site surgery.

Contributing Factors

Performing surgery on the wrong site can have serious consequences for the patient. Patients may be affected emotionally as well as physically from surgery performed on the wrong surgical site. An ineffective surgical site verification procedure can contribute to the incidence of wrong site surgery. Procedure shortcomings might include:
Inadequate patient assessment.
Inadequate medical record review, lack of institutional controls.
Miscommunication among members of the surgical team and the patient.
Exclusion of certain surgical team members, and reliance solely on the surgeon for determining the correct surgical site. 7

Other factors that may contribute to an increased risk of wrong site surgery include:
Having more than one surgeon involved in the procedure,
Performing multiple procedures on multiple parts of a patient during a single surgical encounter,
Unusual time pressures,
Pressure to reduce preoperative preparation time,
Patient characteristics requiring unusual equipment setup or patient positioning,
Failure to include the patient and/or family members/significant others when identifying the correct site, and
Incomplete or inaccurate communication among members of the surgical team.8
   Risk-Reduction Strategies

AORN is in agreement with and suggests the following strategies for developing facility procedures/protocols for identifying the correct surgical site. 9
Involve the patient and/or family members/significant others in identifying the correct site.
Use a specified, clear, unambiguous, indelible method for marking only the correct surgical site.
Specify in individual facility policy and procedure how, when, and by whom the surgical site is to be marked.
Use a verification checklist immediately before surgery that includes the following:

Verbal communication with the patient and/or family members/significant others.
Medical record review, including the face sheet, history and physical, and preoperative assessment.
Review of the informed consent.
Review of all available imaging studies; and direct observation of the marked surgical site.
Verbally verify the correct site with each member of the surgical team.
Use quality control initiatives to monitor compliance with protocol.
   AORN's Position

AORN is committed to promoting identification of the correct surgical site. Using the suggested risk-prevention strategies when developing policies and procedures will reduce the risk of error. As patient advocates, perioperative RNs should communicate with all members of the surgical team to verify the correct surgical site. Individual facility policy should clearly delineate the role and responsibility of the physician and other team members in marking and verifying the correct surgical site.

Definitions

Sentinel event. “A sentinel event is an unexpected occurrence involving death or serious physical or psychological injury, or the risk thereof. Serious injury specifically includes loss of limb or function. The phrase or the risk thereof includes any process variation for which a recurrence would carry a significant chance of serious adverse outcome. Such events are called ‘sentinel’ because they signal the need for immediate investigation and response. 10

Wrong level/part surgery. A surgical procedure that is performed at the correct site, but at the wrong level or part of the operative field. For example, performing a lumbar laminectomy on an unintended intervertebral level immediately adjacent to an intervertebral level with identified pathology. In this type of error, the correct part of the body is prepped and draped, but the surgical procedure is performed on the wrong level of the patient's anatomy. 11

Wrong patient surgery. A misidentification of the patient. This type of error includes procedures that are performed on the wrong patient. 12

Wrong side surgery. A surgical procedure that involves errors on extremities or distinct sides of the body. 13

Wrong site surgery. A broad term that encompasses all surgical procedures performed on the wrong body part or the wrong patient. 14
1. Institute of Medicine, To Err is Human: Building a Safer Health System (Washington, D.C.: National Academy Press, 2000).
2. “ANA code for nurses with interpretive statements: Explications for perioperative nursing,” in Standards, Recommended Practices & Guidelines (Denver: AORN, Inc, 2001) 53-70.
3. “Sentinel events,” in Comprehensive Accreditation Manual for Hospitals: The Official Handbook (Oakbrook Terrace, III: Joint Commission on Accreditation of Healthcare Organizations, November 2000) SE-2. ECRI, “Operating room risk management,” ORRM Surgery 23 (August 2000) 1-2.
4. “Sentinel events,” SE-2.
5. Joint Commission on Accreditation of Healthcare Organizations, “Lessons learned”: Wrong site surgery, in Sentinel Event Alert, no 6. Available from hftp://wwwjcaho.org/edu %5Fpub/sealert/sea6.html. Accessed 3 Jan. 2001.
6. American Academy of Orthopaedic Surgeons, “American Academy of Orthopaedic Surgeons Advisory Statement, Wrong-Site Surgery. ” Available from hftp:/ANww.aaos.org. Accessed 3 Jan. 2001.
7. Joint Commission on Accreditation of Healthcare Organizations, “Lessons learned: Wrong site surgery”; ECRI, “Operating room risk management,” 5-6.
8. Ibid
9. Ibid.
10. “Sentinel events” SE-1.
11. ECRI, “Operating room risk management,” 2.
12. Ilbid.
13. ECRI, “Operating room risk management,” 1.
14. “Sentinel events,” SE-2; ECRI, “Operating room risk management,” 1. Original statement adopted by Board of Directors in February 2001 Ratified by the House of Delegates, Dallas, Tex., in March 2001. Sunset Review: March 2006

What is claimed is:

1. An apparatus for preventing wrong-site surgery comprising;
a storing device for inputting information and medical histories of a patient; and
a tattoo Stencil for marking the information and medical histories of the patient to patient's body.

2. The apparatus of claim 1, wherein the storing device is a digital storage device.

3. A method for preventing wrong-site surgery comprising the steps of:
inputting patient's information, medical history and diagnosis according to checklists from physicians to a storing device;
affixing the data to the patient's body as a tattoo;
transmitting the data from the storing device to a surgeon's electronic device;
comparing the data received by the surgeon to the surgeon's opinion arrived at when the patient attends the pre-operation work-up;
transferring the compared data if confirmed to the surgical team; and
comparing the patient's tattoo to the data transferred to the surgical team as a final check to prevent wrong-site surgery.

4. The method of claim 3, wherein the storing device is a digital storage device.

5. The method of claim 3, wherein the media device is a monitor.

6. The method of claim 3, wherein the check list includes;
displaying basic anatomical detail and concise patient history;
giving a detailed anatomical chart printout; and
providing accesses to the next layer of patient history.
